(19) United States
(12) Patent Application Publication
(10) Pub. No.: US 2008/0125756 A1
(43) Pub. Date: May 29, 2008

(54) Adjustable lock pigtail loop drainage catheter and catheter connector lock

(76) Inventors: Paul Dicarlo, Middleboro, MA (US); Jeffrey M. Cross, Charlestown, MA (US); David S. Cooke, Harvard, MA (US)

Correspondence Address: FAY KAPLUN & MARCIN, LLP, 150 BROADWAY, SUITE 702, NEW YORK, NY, 10038, US

(21) Appl. No.: 11/503,749

(22) Filed: Aug. 14, 2006

(51) Int. Cl. A61M 27/00

(52) U.S. Cl. 604/543; 604/540; 128/898

(57) ABSTRACT

A catheter comprises an elongated body extending from a flexible distal portion to a proximal portion and including a fluid transport lumen extending therethrough and a tensioning member lock collar slidably coupled to the proximal portion for movement relative thereto, the tensioning member lock collar including a connection for coupling the proximal portion of the catheter to a fluid receiving structure in combination with a tensioning member coupled between the flexible distal portion and the tensioning member lock collar so that movement of the tensioning member lock collar proximally along the elongated body draws the tensioning member proximally a first distance and curls the flexible distal portion by a first amount.

### BACKGROUND OF THE INVENTION

Catheters are routinely used to form a semi-permanent path into the body for allowing the drainage of fluids from sites of infection, trauma, or abscess or fluids such as urine which are produced through normal metabolic function. Pigtail catheters are a common type of these drainage catheters, with a distal portion that curls like a pig's tail to maintain the catheter in the desired location.

Pigtail catheters are maintained straight during insertion, and upon deployment are curled into an annular shape. These catheters are especially useful for the drainage of abscesses or of the urinary bladder. The pigtail construction promotes retention of the catheter in the target location (e.g., within the bladder) as the size of the curled distal tip extends across the opening of the vessel through which the catheter was inserted into the location. The distal tips of conventional pigtail catheters are looped by pulling a string or suture extending from the distal tip along the catheter to a manual control at the proximal end and the catheter is then connected to another device (e.g., a collection bag, additional tubing) using, for example, a pair of locking hubs.

### SUMMARY OF THE INVENTION

In one aspect, the present invention is directed to a catheter comprising an elongated body extending from a flexible distal portion to a proximal portion and including a fluid transport lumen extending therethrough and a tensioning member lock collar slidably coupled to the proximal portion for movement relative thereto, the tensioning member lock collar including a connection for coupling the proximal portion of the catheter to a fluid receiving structure in combination with a tensioning member coupled between the flexible distal portion and the tensioning member lock collar so that movement of the tensioning member lock collar proximally along the elongated body draws the tensioning member proximally a first distance and curls the flexible distal portion by a first amount.

### BRIEF DESCRIPTION OF THE DRAWINGS

FIG. 1 is a top plan view showing a catheter according to an embodiment of the invention, with the distal end formed into a pigtail;

FIG. 2 is a side elevation view of the catheter shown in FIG. 1;

FIG. 3 is a top view showing a catheter according to a different embodiment of the invention;

FIG. 4 is a top view of the catheter of FIG. 3 showing a locking arrangement of the catheter and a second device coupled thereto;

FIG. 5 is a cross-sectional view of a suture lock collar used in the catheter of FIG. 3;

FIG. 6 is a perspective view showing an embodiment of a drainage catheter with an adjusting mechanism comprising a thumb wheel according to the invention;

FIG. 7 is a perspective view of a second embodiment of a drainage catheter with an adjusting mechanism comprising a slider ring according to the invention;

FIG. 8 is a perspective view of a third embodiment of a drainage catheter with an adjusting mechanism comprising a spring loaded slide;

FIG. 9 is a perspective view of a further embodiment of a drainage catheter with an adjusting mechanism comprising a threaded lock connector;

FIG. 10 is a cross sectional view showing another embodiment of a drainage catheter with a uniform non-transitioning lumen;

FIG. 11 shows an exemplary embodiment of a permanent catheter connector lock in the locked position;

FIG. 12 shows the catheter connector lock of FIG. 11 in a partially open position;

FIG. 13 shows a second embodiment of a permanent catheter connector lock according to the invention before being locked;

FIG. 14 shows the lock of FIG. 13 in a locked position;

FIG. 15 shows another embodiment of suture lock collar used in a permanent catheter connector lock according to the invention;

FIG. 16 shows a second lock collar used with the suture lock collar shown in FIG. 15;

FIG. 17 a different embodiment of a permanent catheter connector lock according to the invention before being locked; and

FIG. 18 shows the connector lock of FIG. 17 in the closed configuration.

### DETAILED DESCRIPTION

The present invention may be further understood with reference to the following description and the appended drawings, wherein like elements are referred to with the same reference numerals. The invention relates generally to catheters and more particularly to catheters for draining fluids from the body.

The present invention provides a pigtail catheter deployable by applying tension to a suture that is largely internal to the catheter during and after deployment, reducing the risk of infection and of degradation of the suture material over time, as well as reducing chances of leakage. The embodiments of the present invention also provide for a pigtail catheter in which the distal end is automatically curled into a desired pigtail shape as the catheter is joined to another device without requiring the user to perform any additional act. The distance through which the suture is pulled can be further adjusted according to embodiments of the invention, so that the pigtail loop is tightened to a desired degree.

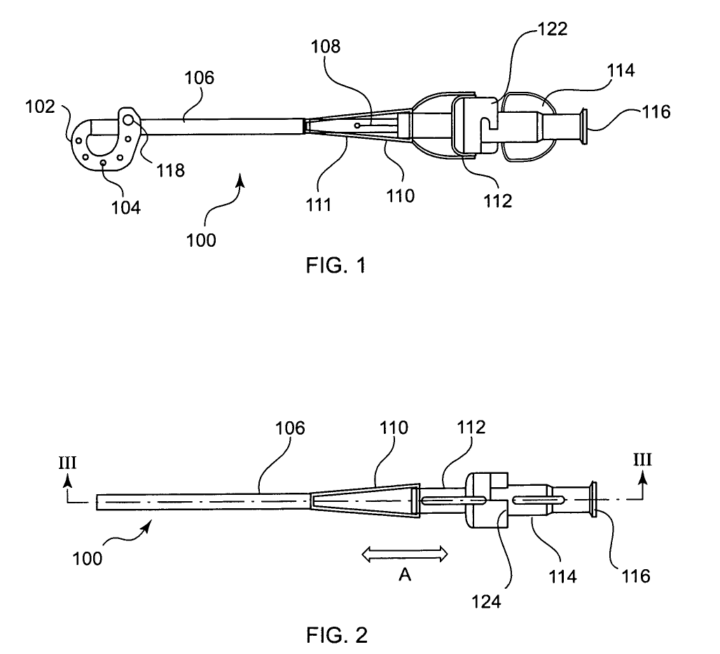

As shown in FIGS. 1-5, a catheter 100 according to an exemplary embodiment of the invention is a drainage catheter, such as a urinary catheter, that is advanced into the bladder or into the renal pelvis to drain urine therefrom. In other embodiments, the catheter 100 may be used to drain abscesses or cysts. As would be understood by those skilled in the art, a size of the catheter 100 is preferably selected to tailor the catheter 100 to a particular application for which it is to be used.

The exemplary catheter 100 comprises a flexible, elongated body 106 terminating at a distal pigtail portion 102 which is biased toward a desired curled shape by, for example, heat setting. The elongate body 106 is generally kept substantially straight during insertion into the site to be drained by, for example, a stiffening member inserted therein. A suture 108 is attached internally near a distal tip 118 of the distal pigtail portion 102 and runs along a lumen 101 extending through the catheter 100 substantially the length of the elongated body 106, to an opening 103 formed in a proximal end of the catheter 100. A plurality of holes 104 are formed through the wall of the distal pigtail portion 102 to fluidly couple the lumen 101 to the external environment so that fluids to be drained fluid from the area surrounding the distal pigtail portion 102 enter the lumen 101 and pass to a separate fluid conduit or reservoir (not shown) attached to the proximal end of the catheter 100. As would be understood by those skilled in the art, the holes 104 may be distributed along any portion of the length of the elongated body 106 to tailor the catheter 100 to any specific application but will generally be distributed along only that portion of the length of the elongated body 106 which, when the catheter 100 is in a desired position within the body, is located within the area to be drained.

A first suture lock collar 112 is slidably received around the proximal portion of the exemplary catheter for movement between a distal, de-coupled position and a proximal, coupled position in which the first suture lock collar 112 may be engaged with a corresponding second lock collar 114 of a mating separate device. This connection is used to fluidly couple the lumen 101 to a corresponding lumen in the separate device. The suture 108 extends through the lumen 101 to an exit orifice 111 through which the suture 108 penetrates the wall of the catheter 100 to couple to the first suture lock collar 112. The exit orifice is preferably located on a portion of the catheter 100 which, when the first suture lock collar 112 is in the distal, de-coupled position, is covered by the first suture lock collar 112. This portion of the catheter 100 is designed to remain outside the body when the distal pigtail portion 102 is in a desired position within the body. Moving the first suture lock collar 112 proximally from the de-coupled position to the coupled position to connect the catheter 100 to the separate device draws the suture 108 proximally, pulling the distal pigtail portion 102 into the desired curved, pigtail shape.

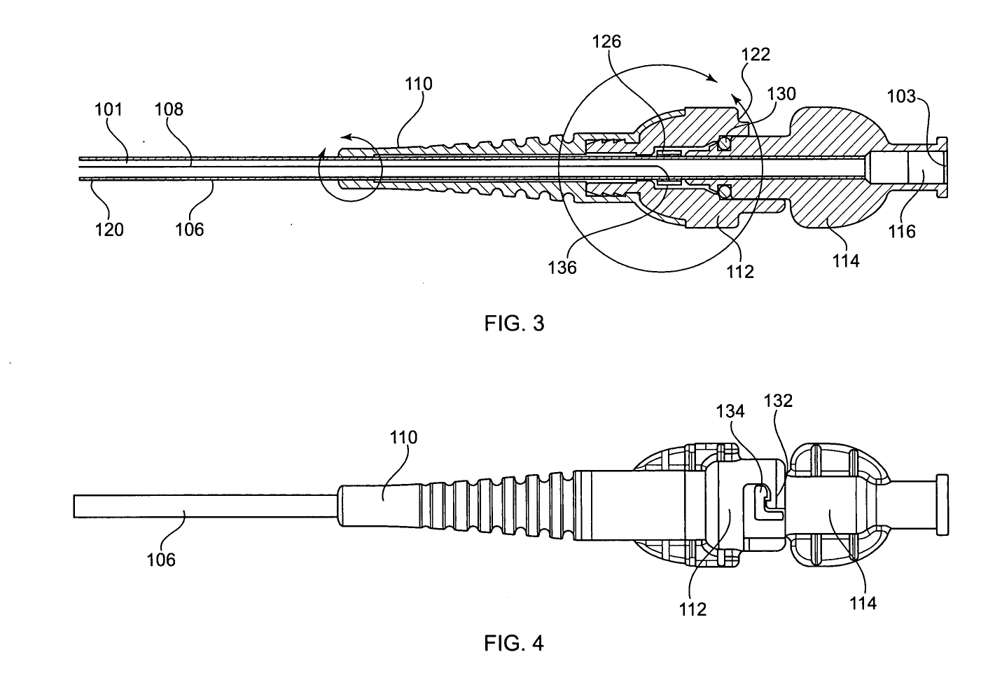

As shown in FIG. 3, and as described above, the first suture lock collar 112 is slidable longitudinally along the elongated body 106 between the coupled and de-coupled positions, and comprises a connection end 122 adapted to form a fluid tight connection via an optional O-ring 130 which is compressed as the second device is pushed onto the catheter 100 to lock the first suture lock collar 112 and the second lock collar 114 to one another. The mating connector includes a connection end 124 which, when the second lock collar forms a connection with the first suture lock collar 112, establishes a fluid connection between the lumen 101 and a drainage bag or other device coupled to the mating connector. As would be understood by those skilled in the art, once the first suture lock collar 112 and the second lock collar 114 are locked in place, the O-ring 130 expands further increasing the difficulty in disassembling the first suture lock collar 112 and the second lock collar 114. Those skilled in the art will understand that the locking hubs 112, 114 may have, for example, a mechanical coupling or friction fit so that they may be easily snapped together to form the connection.

As shown in FIGS. 3-5, the second lock collar 114 of this embodiment includes an O-ring 130 received within a channel near a distal end thereof. The O-ring 130 provides a seal between the first suture lock collar 112 and the second lock collar 114 so that any fluids leaking from the exit orifice 111 are prevented from escaping to the exterior of the first suture lock collar 112 and/or the second lock collar 114. In addition as described above, as the first suture lock collar 112 is pushed proximally into the second lock collar 114, the O-ring 130 is compressed slightly. When the pressure against the O-ring 130 is released it expands to maintain a locking arm 134 of the second lock collar 114 in a locking position within a mating arm 132 of the first suture lock collar 112.

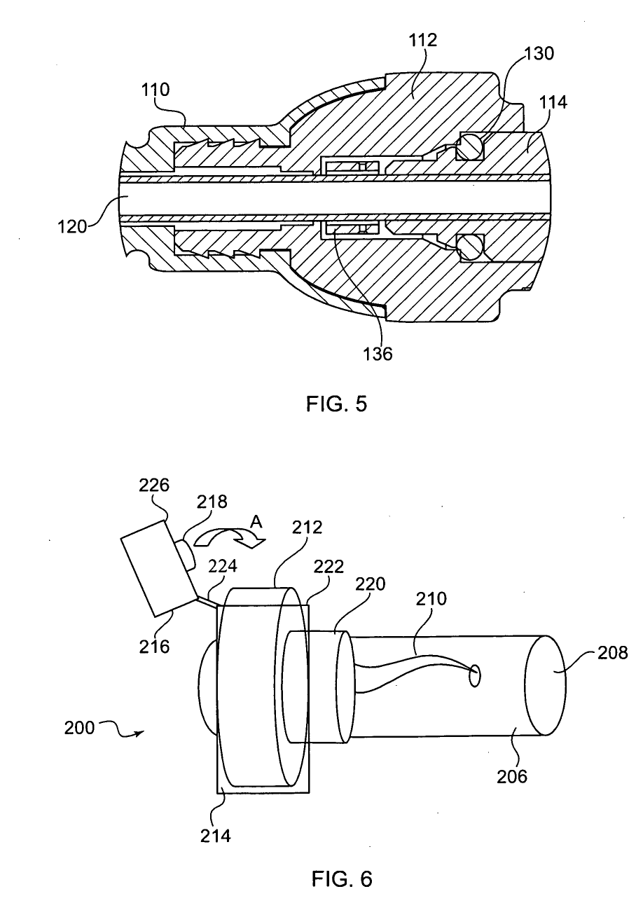

In addition, FIGS. 3 and 5 show the suture collar 136 to which the suture 108 is coupled. Those skilled in the art will understand that, as the suture collar 136 is received within the first suture lock collar 112, movement of the first suture lock collar 112 proximally draws the suture collar 136 and the suture 108 attached thereto proximally. The bias of the distal pigtail portion 102 to the curved position prevents the suture collar 136 from moving proximally out of the first suture lock collar 112. Additional details of the locking arm 134 and mating arm 132 may be provided to further secure the connection, as will be described in greater detail below.

As shown in FIG. 1, a short segment of the suture 108 extends out of the elongated body 106 through the exit orifice 111 to the first suture lock collar 112. As described above, when the first suture lock collar 112 is in the distal, de-coupled position, the first suture lock collar 112 completely covers this exposed portion of the suture 108. Even when the first suture lock collar 112 is moved proximally to the coupled position away from the exit orifice 111, only the portion of the suture 108 extending from the exit orifice 111 to the distal end of the first suture lock collar 112 is uncovered. Thus, the length of the suture 108 exposed to the environment outside the catheter 100 is minimized and is less than the distance the first suture lock collar 112 is able to translate longitudinally along the elongated body 106, as will be described below.

A flexible, strain relief section 110 or sheath may also be provided around the proximal portion of the catheter 100 to form a seal around the exposed portion of the suture 108 to prevent fluid leakage from the orifice 111 from contaminating the surfaces of the catheter 100 which are handled by health care professionals, etc. The flexible, strain relief section 110 may, for example, be formed as a shell-like structure a distal end of which creates a fluid tight seal around a portion of the elongated body 106 immediately distal to a distal end of the first suture lock collar 112 when the first suture lock collar 112 is in the de-coupled position.

A proximal end of the strain relief section 110 is preferably coupled to the distal end of the first suture lock collar 112 so that, as the first suture lock collar 112 is moved proximally to the coupled position, the strain relief section 110 is drawn proximally to cover the exposed portion of the suture 108 while maintaining a seal around the entire area exposed to the suture 108 and any fluids that may leak from the exit orifice 111. Thus, drawing the first suture lock collar 112 proximally to couple the catheter 100 to a separate device draws the distal pigtail portion 102 into the desired curved, pigtail shape and pulls the strain relief section 110 proximally to ensure that the suture 108 is completely internal to the catheter 100 and is not exposed to the outside environment.

As shown in the enlargements of FIGS. 3 and 5, the suture 108 is attached to the suture collar 136 which is received within the first suture lock collar 112 so that when the first suture lock collar 112 is moved proximally, the suture collar 136 and the suture 108 are pulled along with it. As described above, a locking collar of a conventional pigtail catheter is generally translated proximally after insertion, to connect the catheter to another medical device, for example, a drainage bag. In contrast, as the catheter 100 according to the invention is translated to effectuate the mechanical and fluid connection to a separate device, this translation of the first suture lock collar 112 draws proximally the suture collar 136 and the suture 108 coupled thereto, curling the distal pigtail portion 102 into the desired pigtail shape. This occurs without the user having to perform the separate step required with prior devices.

The use of the catheter 100 is described below with reference to FIGS. 1 and 2. The insertion of the catheter 100 proceeds in a generally conventional manner, with the first suture lock collar 112 in the distal, de-coupled position spaced from the proximal end of the catheter 100 toward the distal pigtail portion 102. At this point the second locking collar 114 has not yet been connected to the first suture lock collar 112. After the catheter 100 has been properly positioned, e.g., with the distal pigtail portion 102 extending past an opening from a small diameter vessel through which the catheter 100 was introduced into a larger organ, the user slides the first suture lock collar 112 proximally in the direction of arrow A, bringing the first suture lock collar 112 nearer to the proximal end of the catheter 100 for connection to the second lock collar 114.

As described above, this proximal motion of the first suture lock collar 112 applies tension to an actuation member and the suture 108, which in turn pulls the distal tip 118 inward and curls the distal pigtail portion 102 to form the pigtail shape within the larger hollow organ. The curled distal pigtail portion 102 then provides a check to removal of the catheter 100 from the large organ as a diameter of the curled shape of the distal pigtail portion 102 is preferably selected to be larger than that of the vessel via which the catheter 100 was inserted into the organ. Thus, no separate action needs to be performed to secure the catheter 100 in place, as the pigtail shape is formed automatically when the first suture lock collar 112 is connected to the second lock collar 114. Then, when the first suture lock collar 112 has been connected to the second lock collar 114, it is no longer able to move distally in the direction of the arrow A and the tension remains on the suture 108 which effectively locks the distal pigtail portion 102 in the curled, pigtail shape preventing unintentional removal of the catheter 100 from the desired location.

In many applications it is desirable to control the shape of the pigtail formed at the distal end of the catheter when the suture is pulled proximally during connection of the suture lock collar to the second lock collar. For example, as shown in FIG. 1, the distal pigtail portion 102 may take a fully curled shape that approximates a circular loop. Alternatively, for other applications it may be beneficial to maintain the distal pigtail portion 102 in a less curled shape, which may range from being substantially straight to having a more or less pronounced semi-circular outline.

The embodiments of the present invention provide to the user a system to select an amount of curling, or curvature of a distal flexible portion of a catheter after a suture lock collar thereof has been moved proximally to connect with the luer. The shape of the flexible distal portion after movement of the suture lock collar, referred to as the first loop configuration, may be a minimally curved loop. In one exemplary embodiment, an adjusting mechanism is provided that lets the user select an additional distance through which the suture collar 136 is pulled after a given movement (called the throw) of the first suture lock collar 112. In this case, the throw of the mechanism can be reduced, since it is not exclusively responsible for drawing the suture proximally. Those skilled in the art will understand that the adjusting mechanism may be formed as a part of the first suture lock collar 112. Alternatively, the adjusting mechanism may replace the suture collar 136.

According to the present invention, the draw applied to the suture does not have to be directly coupled to the throw of the suture lock collar. Instead, tension may be applied to the suture entirely by the adjusting mechanism, or by a combination of the translation of the suture lock collar and the adjusting mechanism. In the exemplary embodiment shown in FIG. 6, the adjustable lock pigtail loop 200 has an adjusting mechanism that comprises a suture ring 220 operatively connected to the suture 210. For example, the suture 210 may be attached to the surface of the suture ring 220, and may be wrapped thereon as on a spool as the suture ring 220 is rotated about the axis of the catheter shaft 206. The suture 210 may extend proximally on the inside of the catheter shaft 206 from a distal end 208, and is placed under tension and pulled proximally when the suture ring 220 rotates.

According to an exemplary embodiment of the invention, the suture ring 220 may be rotated by engaging a thumb wheel 212 coupled to the suture ring 220 so that both rotate together. As would be understood by those skilled in the art, the thumb wheel 212 may be ergonomically designed with a width and diameter appropriate to typical hands (or with thumb wheels 212 of various sizes), so that it is easy and comfortable to turn manually while performing a procedure. During the procedure, for example after the suture lock collar 212 has been connected to the second lock collar 214, the thumb wheel 212 may be rotated in the direction of arrow A to wrap more of the suture 210 around the suture ring 220, further pulling the distal pigtail portion 102 into a curl or in a direction opposite that of arrow A to unwrap the suture 210 from the suture ring 220 to allow the distal pigtail portion 102 to straighten to a desired degree. The more the thumb wheel 212 is rotated, the more pronounced is the curling which results. Thus, a desired second loop configuration of the flexible distal pigtail portion 102 may be formed, which may comprise a substantially fully formed loop. Those skilled in the art will understand that the suture 210 may alternatively be tensioned or loosened to adjust the curvature of the distal pigtail portion 102 by twisting and untwisting the suture 210.

In the exemplary embodiment shown in FIG. 6, the thumb wheel 212 and the suture 210 are mounted on the catheter shaft 206, separately from the suture lock collar 212. In other embodiments, the thumb wheel 212 and suture ring 220 assembly may be connected to a slidable locking device such as the first suture lock collar 112 shown in FIG. 1. In this case, the suture ring 220 translates together with the first suture lock collar 112, providing an initial tensioning of the suture 210 when a connection is made between the catheter body 106 and the luer 116.

The initial tensioning may, for example, place the distal pigtail portion 102 in a first loop configuration with a relatively minimal curvature of the flexible distal pigtail portion 102. Further actuation of the thumb wheel 212 in the direction of the arrow A further tensions the suture 210 and draws the distal pigtail portion 102 into a second configuration (e.g., a fully formed pigtail loop). The throw of the first suture lock collar 112 in this configuration may be kept to the minimum distance consistent with making the connection with the second lock collar 114, as the tensioning of the suture 210 may be further adjusted using the thumb wheel 212.

The adjustable lock pigtail loop 200 may also comprise a thumb wheel guard 214 adapted to prevent unintentional movement of the thumb wheel 212 during the procedure. The thumb wheel guard 214 may enclose a majority of the thumb wheel 212, and may comprise an opening 222 having dimensions suitable for the user to operate the adjusting mechanism, for example, using the thumb. The exemplary thumb wheel guard 214 comprises a thumb wheel cover 216 covering the opening 222 when there is no need to use the adjusting system. For example, the cover 216 may be attached to the thumb wheel guard 214 via a hinge 224 and a snap or latch 226. A friction lock 218 may be included in the thumb wheel cover 216 to frictionally engage the surface of the thumb wheel 212, and prevent its rotation once the loop has been properly formed or to maintain a current curvature of the distal pigtail portion 102 at any time. For example, the friction lock 218 may include a mechanical lock and/or may be made of silicone or of another material providing high friction.

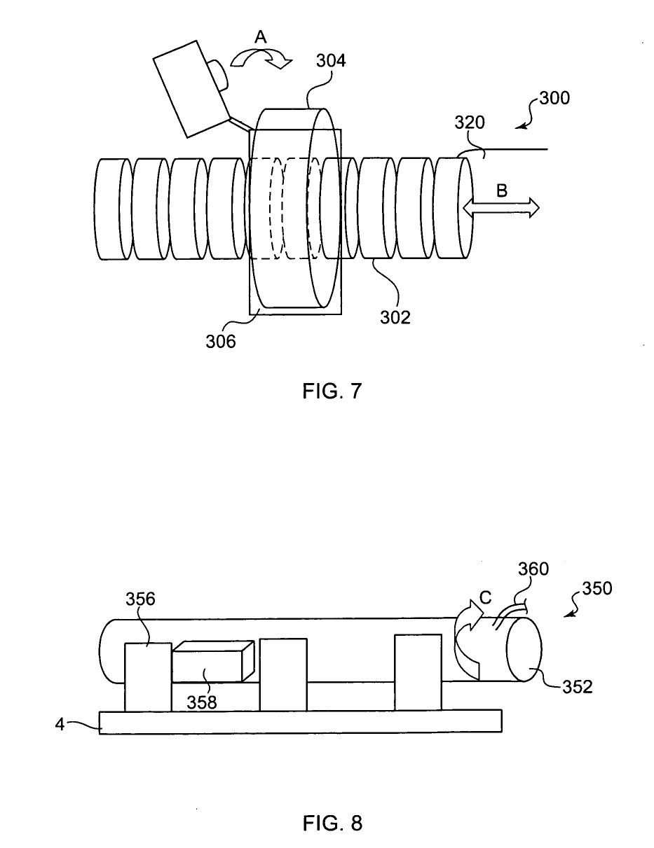

As shown in FIG. 7, a mechanism 300 for adjusting suture tension according to an alternative embodiment of the present invention changes the rotational action of the thumb wheel 304 into a linear motion. In this embodiment, the suture 320 is attached to a linear suture guide 302 placed on the body of the catheter 100. The linear suture guide 302 is coupled to the thumb wheel 304 such that, as the thumb wheel 304 is rotated in the direction of arrow A, the linear suture guide 302 translates longitudinally in the direction of the arrow B.

For example, the inner surface of the thumb wheel 304 may cooperate with the outer surface of the slider suture ring 302 to achieve the desired motion. The thumb wheel 304 may be offset relative to the slider suture ring 302, or surface sculpturing of either or both of the two components may be utilized to obtain the relative motion therebetween. The mechanism 300 according to this embodiment of the invention reduces tangling of the suture while it is drawn tight further simplifying the procedure. For example, as would be understood in the art, the mechanism 300 may be similar to a worm gear engaging a slider or a threaded rod and a nut where the rotating member is held in place longitudinally while the member it engages moves along the threading.

Additional embodiments of the present invention may be devised, that utilize different mechanisms to adjust the tension of the suture as required. In general, these embodiments allow the use of an adjustable lock for a pigtail loop used in a drainage catheter that has a short throw. In these embodiments, locking the adjustable lock forms a fluid and mechanical connection between the two devices, but only partially draws the suture so that the pigtail is coiled to a minimally curved position. If desired, a tighter pigtail loop may be achieved by actuating an adjusting mechanism that may be mounted on the adjustable lock or separate therefrom.

As shown in FIG. 8, an adjusting mechanism 350 according to a further embodiment of the invention comprises a linear slide 352 with a spring loaded lever 358. The linear slide 352 cooperates with a locking element 354 having a plurality of locking tabs 356 that protrude therefrom, and that interact with the spring loaded lever 358. The suture 360 is attached to the linear slide 352, so that when the linear slide 352 translates longitudinally in the proximal direction, the suture 360 is tensioned forming the pigtail. During use, moving the spring loaded lever into another notch between locking tabs 356 tightens the loop.

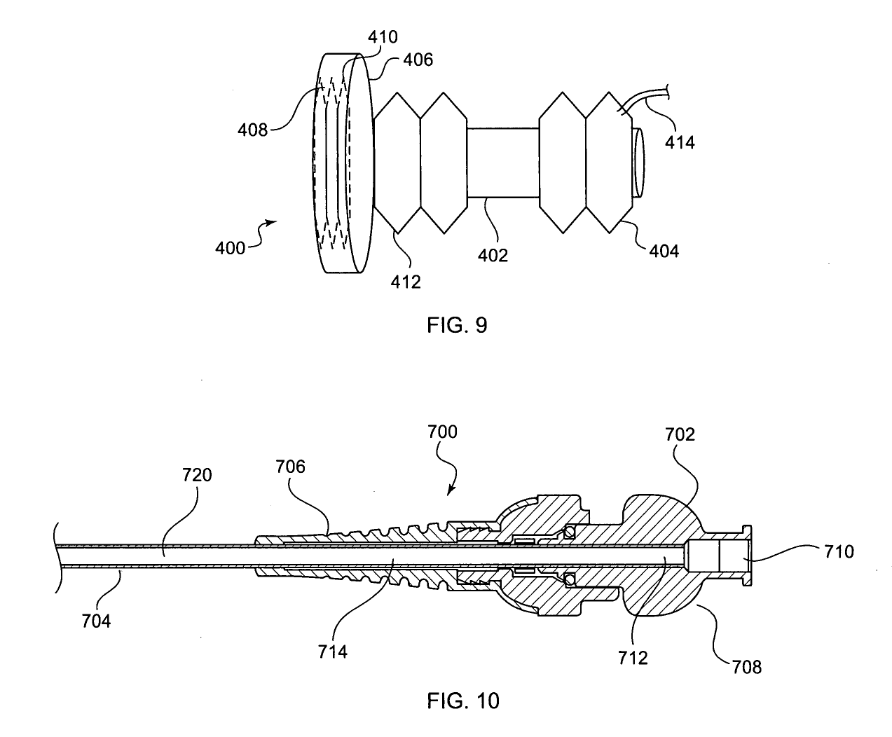

As shown in FIG. 9, an adjustable lock 400 according to a further embodiment of the invention comprises a lock bypass 402 that has a proximal thread connector to which the suture 414 is attached. The lock bypass 402 enables a user to achieve a desired amount of tightening of the suture 414 with a reduced amount of rotation of a thumb wheel 410. Internal threads 408 of the thumb wheel 410 engage ridges 412 of the proximal connector 404, so that curvature of a pigtail loop attached to the suture 414 can be adjusted from a first, minimally curved shape to any desired curvature up to a substantially fully formed loop. In this embodiment, the proximal thread connector 404 and the distal lock connector 406 are initially brought together to form a mechanical and fluid connection and the thumb wheel 410 is then rotated to adjust the tension on the suture 414 and, consequently, to achieve a desired curvature (shape) of the pigtail loop. As would be understood by those skilled in the art, the thumb wheel 410 may be biased to ensure that the ridges 412 are continuously engaged by the internal threads 408.

As described above, typical drainage catheters include an elongated portion with a lumen extending therethrough to a hub which is connected to another device. Because the overall flow passage extends through multiple components and the joints between these components, it can be non-uniform, including restrictions negatively affecting the flow rate and increasing the time required to drain a given amount of fluid.

Exemplary embodiments of the catheter according to the invention comprise an elongated body incorporating an internal suture which exits the catheter at a location separate from the hub. Since the exit port for the suture is not formed in the hub, the hub may be designed to incorporate a flow passage that is more uniform and substantially similar to the lumen extending through the elongate body of the catheter, reducing flow restrictions as compared to conventional hubs and increasing the flow rate therethrough. Specifically, the hub according to one exemplary embodiment is sized so that the elongated body of a drainage catheter may be inserted into the distal end of the hub and advanced therethrough to the proximal end of the hub. The lumen of the catheter thus extends through a large portion of the hub, resulting in a more uniform flow path with fewer discontinuities and restrictions.

As shown more clearly with reference to FIG. 10, the exemplary catheter 700 comprises a hub 702 and an elongated body 704 connected thereto. The hub 702 comprises a catheter receiving portion 714 that extends through the body of the hub 702 to a location adjacent to a proximal end 710 thereof. The proximal end 712 of the catheter elongated body 704 extends into the catheter receiving portion 714, so that the catheter lumen 720 forms a flow passage extending substantially from the distal tip of the elongated body 704 to the proximal end 710 of the hub 702. Since there are no transitions between different components along this portion of the overall flow path—from the distal end of the catheter 700 to the proximal end 710 of the hub 702, flow restrictions in this portion of the flow path are minimized.

The hub 702 according to this embodiment includes a connection portion 708 for mechanically attaching to a connection portion 706 of the elongated body 704. As would be understood by those skilled in the art, the connection portion 708 may comprise any suitable known conventional mechanical attachment. The proximal end 712 of the elongated body 704 extends proximally past the connection portion 706, so that it may be advanced into the catheter receiving portion 714 of the hub 702. In one embodiment, the proximal end 712 may be fluidly connected directly to an external device, such as a receiving bag, additional tubing, etc. Alternatively, a portion of the proximal end 710 of the hub 702 may be included in the overall flow path to the external device.

Standardized connectors such as luers which are configured with a tapered projection on one component and a mating conically shaped element on the other component, have often been utilized to attach fluid conduits. However, a locking system that prevents disassembly of the catheter system by the patient or any other unauthorized person may be desired for certain applications as such disassembly may reduce the efficacy of the procedure or cause injuries or other problems.

Embodiments of the present invention incorporate a J-lock to provide such a ‘permanent’ catheter lock which is not easily disassembled by unauthorized personnel. J-Locks are well suited for applications such as, for example, biopsy needle catheters, which include components subject to sudden motions. In addition, an O-ring may be included for some applications. A conventional J-lock is shown in FIG. 4, with a locking arm 134 cooperating with a mating arm 132 to form a connection. Such conventional J-locks are equally easily connected and disconnected. In contrast, the J-lock connections according to the present invention incorporate features making them difficult to disassemble while remaining easy to assemble. The geometry of the exemplary connection provides a large resistance to the movement of the parts in an unlocking direction with a much lower resistance to the movement of the parts in a locking direction.

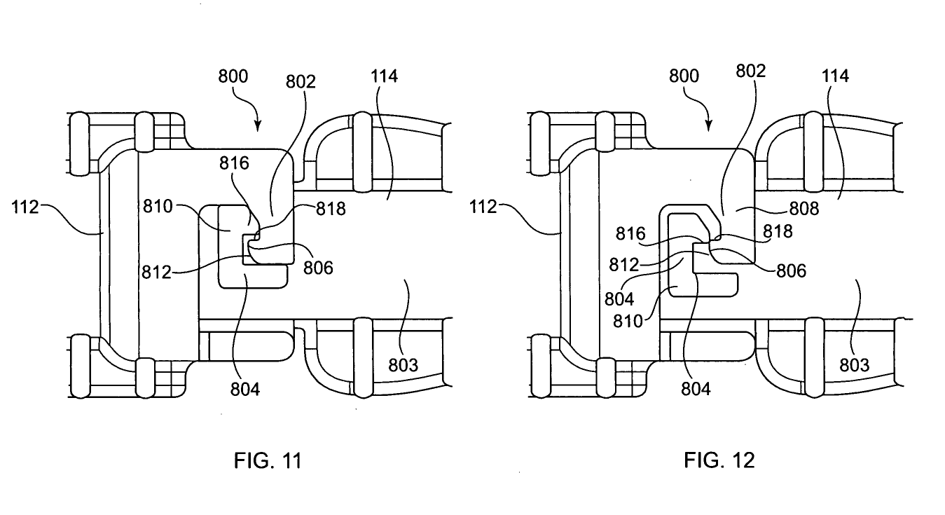

As shown in FIGS. 11 and 12, a J-Lock connector 800 according to a first exemplary embodiment of the invention comprises a mating arm 802 extending from the first suture lock collar 112 to cooperate with a locking arm 804 extending from a second lock collar 114 of a mating connector 803. According to the invention, a uni-directional ramp feature makes it more difficult to disconnect the first suture lock collar 112 and the second lock collar 114 than it is to connect them. FIG. 11 shows the J-lock connector 800 when engaged while FIG. 12 shows the J-lock connector 800 partially disengaged, with the ramp feature thereof preventing further disengagement. In the exemplary embodiment, a ramp 806 is formed on a surface perpendicular to the axis of the first suture lock collar 112. More specifically, the ramp 806 is formed on the surface 812 of the mating arm 802.

As the first suture lock collar 112 and the second lock collar 114 are twisted together, the mating lock 816 of the locking arm 810 rides over the ramp 806, and then falls into the relief 808 to fully engage the connector 800 with the second lock collar 114. A step 818 is formed at the edge of the ramp 806 to prevent interference between the locking arm 804 and the corner of the mating arm 802. As described above, in one exemplary embodiment, an O-ring is compressed as the second lock collar 114 is pushed onto the first suture lock collar 112 urging the first suture lock collar 112 axially away from the second lock collar 114 increasing the axial force necessary to pull the first suture lock collar 112 and the second lock collar 114 sufficiently apart for the locking arm 810 to clear the step 818 and ramp 806 allowing the first suture lock collar 112 and the second lock collar 114 to come apart.

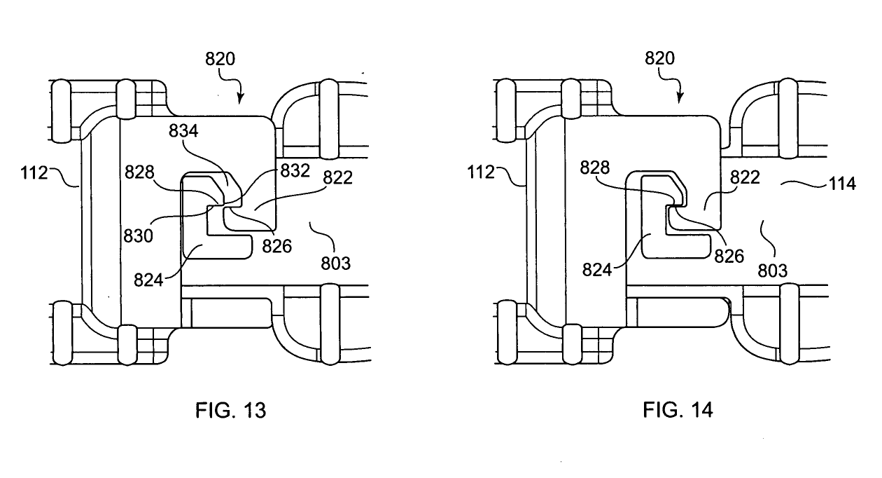

As shown in FIGS. 13 and 14, a connector 820 according a second embodiment of the invention includes a ramp 826 extending substantially parallel to a longitudinal axis of the connector 820 at the tip of a mating arm 822 of the first suture lock collar 112. A corresponding ramp 828 is formed at a tip of the locking arm 824, extending from the second lock collar 114.

As the first suture lock collar 112 and the second lock collar 114 are rotated to mate the ramps 826, 828 together, they ride easily over each other until the ends of projections 832, 834 pass one another. At this point, the ramps 826, 828 hook into each other as the O-ring pushes the connector 820 and the ramp 826 distally and the second lock collar 114 and the ramp 828 proximally relative to one another. At this point, the locking arm 824 enters the relief 834 and secures the first connector 820 to the second lock collar 114. After the first suture lock collar 112 and the second lock collar 114 are joined as shown in FIG. 14, perpendicular surfaces 830, 832 of the ramps 826, 828, respectively, face and abut one another, substantially parallel to one another effectively preventing the ramps 826, 828 from sliding past one another. Thus, the first connector 820 can not be removed from the second lock collar 114 without causing the structural failure of the material of the surfaces 830, 832 reducing the likelihood that such a connector 820 would be separated from a second connector including a second lock 114 as shown.

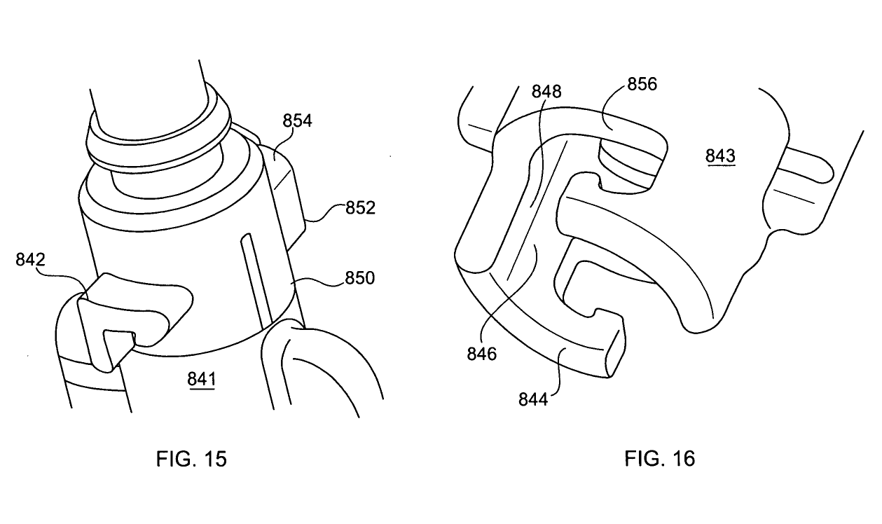

FIGS. 15, 16 show a third embodiment of the permanent J-lock according to the invention. In this exemplary lock, the ramps are located on inside diameter surfaces of the connectors. As shown, the collar 841 comprises a locking arm 842 and a ramp 850 with a sloping surface 854 and a perpendicular surface 852. The corresponding collar 843 comprises a mating arm 844 and a ramp 848. The ramp 846 comprises a sloping surface 848 and a perpendicular surface 856. When the collars 841, 843 are joined, the sloping surfaces 854, 843 of the ramps 850, 846, respectively, slide over one another to abutting against one another. Twisting the collars 841, 843 in an unlocking direction, brings the perpendicular surfaces 852, 856 into an opposing relationship preventing the ramps 846 and 850 from sliding past one another so that the collar 841 cannot be unlocked from the collar 843 unless sufficient torsional force is applied to cause failure of the material.

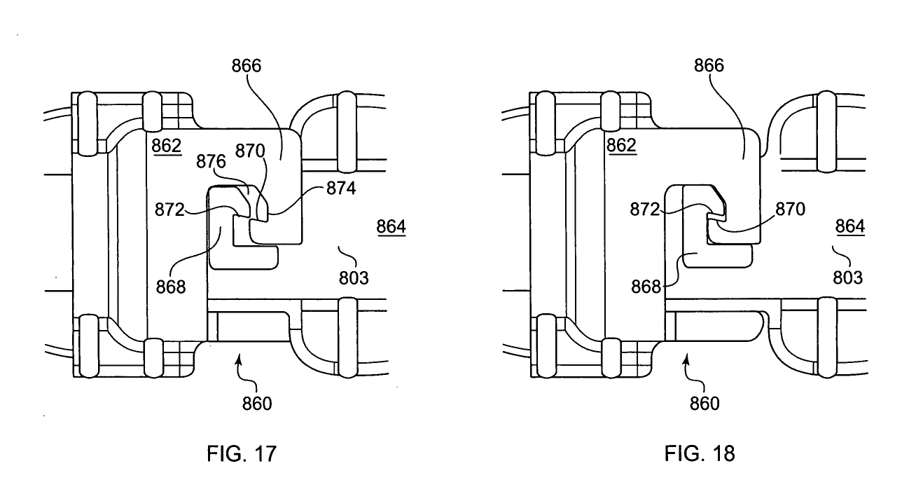

As shown in FIGS. 17 and 18, a permanent J-lock 860 according to yet another embodiment of the invention comprises a suture lock collar 862 with a mating arm 866 adapted to connect to a second lock collar 864 having a locking arm 868. The mating surfaces 870 and 872 respectively of the arms 866, 868 in this exemplary embodiment are angled relative to the axis of the J-lock 860 and the relief opening 876 comprises a projection 874 that increases an interference with the locking arm 868 as the components are joined. The projection 874 urges the angled surfaces 870, 872 against one another resisting the unlocking of the suture lock collar 862 from the second lock collar 864.

Those of skill in the art will understand that the ramps and angled surfaces described above may be formed on the inner and/or outer diameter surfaces of the suture lock collar and of the second lock collar, rather than on arms extending therefrom. In those embodiments, the two components do not need to be twisted to be locked together, but rather the locking features may slide over each other when the components are pushed together axially. A similar level of permanent locking thus is achieved without twisting the components, which may simplify the use of the device in certain procedures.

The present invention has been described with reference to specific embodiments, and more specifically to a catheter connector. However, other embodiments may be devised that are applicable to other medical devices that use fluid connections. Accordingly, various modifications and changes may be made to the embodiments, without departing from the broadest spirit and scope of the present invention as set forth in the claims that follow. The specification and drawings are accordingly to be regarded in an illustrative rather than restrictive sense.

What is claimed is:

1. A catheter comprising:
an elongated body extending from a flexible distal portion to a proximal portion and including a fluid transport lumen extending therethrough;
a tensioning member lock collar slidably coupled to the proximal portion for movement relative thereto, the tensioning member lock collar including a connection for coupling the proximal portion of the catheter to a fluid receiving structure; and
a tensioning member coupled between the flexible distal portion and the tensioning member lock collar so that movement of the tensioning member lock collar proximally along the elongated body draws the tensioning member proximally a first distance and curls the flexible distal portion by a first amount.

2. The catheter according to claim 1, further comprising an adjusting mechanism to further draw the tensioning member proximally to further curl the flexible distal portion to a desired degree.

3. The catheter according to claim 1, wherein the adjusting mechanism is mounted on the tensioning member lock collar.

4. The catheter according to claim 1, wherein the adjusting mechanism comprises a thumb wheel rotatable to wrap the tensioning member therearound and draw a distal end of the tensioning member proximally.

5. The catheter according to claim 4, wherein the thumb wheel rotates about a longitudinal axis of the catheter.

6. The catheter according to claim 4, further comprising a thumb wheel guard.

7. The catheter according to claim 4, further comprising a thumb wheel friction lock.

8. The catheter according to claim 1, further comprising a tensioning member ring coupled to the thumb wheel, rotation of the thumb wheel spooling the tensioning member around an outer surface of the tensioning member ring.

9. The catheter according to claim 1, wherein the adjusting mechanism comprises a thumb wheel which, when rotated, longitudinally translates a slider element connected to the tensioning member.

10. The catheter according to claim 1, further comprising a linear slide cooperating with a spring loaded lever to lock the linear slide in a selected longitudinal position.

11. The catheter according to claim 1, further comprising a threaded proximal connector adapted to engage threads of a distal thread connector of the fluid receiving structure.

12. The catheter according to claim 1, wherein movement of the tensioning member lock collar to lock the catheter to the fluid receiving structure draws the tensioning member proximally to a degree corresponding to the formation of a minimum loop of the flexible distal portion.

13. The catheter according to claim 12, wherein the adjusting mechanism further draws the tensioning member to form a full loop of the flexible distal portion.

14. The catheter according to claim 1, wherein the proximal portion extends into a catheter receiving portion of the fluid receiving structure to form a substantially uniform fluid transport lumen therethrough.

15. The catheter according to claim 1, further comprising a mating arm of the tensioning member lock collar for engaging a locking arm of the fluid receiving structure.

16. The catheter according to claim 15, wherein the mating arm includes a unidirectional ramp adapted to cooperate with a unidirectional ramp of the locking arm to form a permanent connection therebetween.

17. The catheter according to claim 15, wherein the mating arm includes a sloping surface and a perpendicular surface, the perpendicular surface impeding a disconnecting motion of the tensioning member lock collar relative to the fluid receiving structure.

18. The catheter according to claim 15, wherein the unidirectional ramp is substantially perpendicular to a longitudinal axis of the tensioning member lock collar.

19. The catheter according to claim 15, wherein the unidirectional ramp is substantially parallel to a longitudinal axis of the tensioning member lock collar.

20. The catheter according to claim 1, further comprising unidirectional ramps disposed on inner surfaces of the tensioning member lock collar for cooperating with corresponding unidirectional ramps of the fluid receiving structure.

21. The catheter according to claim 15, wherein the unidirectional ramps allow rotation of the tensioning member lock collar in a locking direction, and impede rotation in an unlocking motion.

22. A method for deploying a catheter, comprising:
inserting a flexible distal portion of the catheter into a patient; and
sliding a tensioning member lock collar over a proximal portion of an elongated body of the catheter to couple to a second lock collar of a fluid receiving structure, sliding the tensioning member lock collar over the elongated body moving an actuation member to pull the flexible distal portion into a first loop configuration.

23. The method according to claim 22, further comprising actuating an adjusting mechanism to further pull the flexible distal portion into a second loop configuration.

24. The method according to claim 23, further comprising manually actuating a thumb wheel of the adjusting mechanism to further pull the flexible distal portion.

25. The method according to claim 24, further comprising displacing a thumb wheel cover of a thumb wheel guard to actuate the thumb wheel.

26. The method according to claim 23, further comprising engaging a friction lock after actuating the adjusting mechanism thus immobilizing it.

27. The method according to claim 23, wherein the adjusting mechanism comprises a mechanism such that rotating a thumb wheel results into translation of a slide connected to the tensioning member.

28. The method according to claim 23, wherein the adjusting mechanism comprises actuating a spring loaded lever cooperating with locking tabs to limit translation of a slide connected to the tensioning member.

29. The method according to claim 23, further comprising rotating a threaded thumb wheel of the adjusting mechanism to translate a threaded proximal connector thereby pulling the tensioning member.

30. The method according to claim 23, further comprising manually actuating the adjusting mechanism to pull the flexible distal portion into a substantially fully formed loop.

31. The method according to claim 30, further comprising locking the adjusting mechanism to prevent actuation.

32. The method according to claim 22, further comprising advancing a proximal portion of the catheter into a catheter receiving portion of the fluid receiving structure.

33. The method according to claim 22, further comprising rotating the tensioning member lock collar relative to the second lock collar to engage corresponding unidirectional ramps thereof, the unidirectional ramps impeding unlocking motion of the tensioning member lock collar.

34. The method according to claim 33, wherein rotating the tensioning member lock collar comprises sliding past one another opposing sloping surfaces of the unidirectional ramps in a locking motion, and placing perpendicular surfaces of the unidirectional ramps in abutting relationship to prevent unlocking motion thereof.

35. The method according to claim 22, further comprising locking the tensioning member lock collar to the second lock collar to prevent separation thereof.

36. An adjusting mechanism to selectively curl a flexible distal portion of a catheter, comprising:
a tensioning member having a distal end connected to the flexible distal portion of the catheter;
a thumb wheel with a tensioning member ring attached to a proximal end of the tensioning member, rotation of the thumb wheel about a longitudinal axis of the catheter drawing the tensioning member proximally to curl the flexible distal portion of the catheter;
a thumb wheel guard preventing inadvertent rotation of the thumb wheel; and
a lock selectively immobilizing the thumb wheel.

37. The adjusting mechanism according to claim 36, further comprising:
a slide operatively connected to the tensioning member;
a mechanism translating rotational motion of the thumb wheel to linear motion of the slide relative to a longitudinal axis of the catheter.

38. The adjusting mechanism according to claim 37, wherein internal threads of the thumb wheel engage outer ridges of a proximal thread connector of the catheter to move the proximal thread connector relative to the longitudinal axis as the thumb wheel is rotated.

39. The adjusting mechanism according to claim 37, further comprising a spring loaded arm and corresponding locking tabs cooperating to limit translation of the slide.

40. The adjusting mechanism according to claim 36, wherein the thumb wheel and the tensioning member ring are disposed on a tensioning member lock collar slidable along the catheter to engage a fluid receiving structure to be coupled to the catheter.

41. A catheter comprising:
an elongated body extending from a flexible distal portion to a proximal portion and including a fluid transport lumen extending therethrough;
a lock collar coupled to the proximal portion, including a connection for coupling the proximal portion of the catheter to a fluid receiving structure;
a tensioning member having a distal portion connected to the flexible distal portion and a proximal portion coupled to the lock collar;
a proximal end of the proximal portion extending beyond the lock collar to engage a catheter receiving portion of the fluid receiving structure so that the fluid transport lumen extends from the flexible distal portion to a lumen proximal end couplable directly to the fluid receiving structure.

42. The catheter according to claim 41, wherein the lock collar is moved along an axis of the catheter as it is coupled to a fluid receiving structure to curl the flexible distal portion of the catheter.

43. The catheter according to claim 41, wherein the lock collar is slidably coupled to the proximal portion of the catheter.

44. The catheter according to claim 41, wherein the fluid transport lumen is substantially free of flow restrictions from a distal end of the catheter to the lumen proximal end.

45. The catheter according to claim 41, wherein the fluid transport lumen is formed of a single component.

46. The catheter according to claim 41, further comprising a linkage between the lock collar and the proximal end of the tensioning member, such that sliding the lock collar proximally relative to the elongated body draws the tensioning member proximally.

47. The catheter according to claim 41, further comprising unidirectional ramps of the lock collar cooperating with corresponding features of the fluid receiving structure to prevent an unlocking movement of the lock collar.

48. The catheter according to claim 47, further comprising unidirectional ramps protruding from mating arms of the lock collar to engage corresponding unidirectional ramps of locking arms of the fluid receiving structure.

49. The catheter according to claim 47, wherein the unidirectional ramps of the lock collar comprise a sloping surface for sliding contact with the corresponding feature and a perpendicular surface to abut against the corresponding feature and prevent relative movement thereof.
